(19) United States
(12) Patent Application Publication
(10) Pub. No.: US 2026/0144828 A1
(43) Pub. Date: May 28, 2026

(54) USE OF LACTICASEIBACILLUS RHAMNOSUS FMBL L23004 CNN IN THE PREPARATION OF MEDICAMENT FOR PREVENTING OR TREATING DIABETES MELLITUS

(71) Applicant: SHIHEZI UNIVERSITY, Shihezi City (CN)

(72) Inventors: Hailong Sun, Shihezi City (CN); Yongqing Ni, Shihezi City (CN); Huimin Zhang, Shihezi City (CN)

(21) Appl. No.: 19/367,870

(22) Filed: Oct. 24, 2025

(30) Foreign Application Priority Data
Nov. 28, 2024 (CN) 202411727413.X

(51) Int. Cl. A61K 35/744; A61P 1/14; C12N 1/20; C12R 1/01

(52) U.S. Cl. CPC A61K 35/744; A61P 1/14; C12N 1/205; C12R 2001/01

(57) ABSTRACT

The present disclosure relates to a use of Lacticaseibacillus rhamnosus FMBL L23004 CNN in the preparation of a medicament for preventing or treating diabetes mellitus. The Lacticaseibacillus rhamnosus FMBL L23004 CNN was deposited with the China Center for Type Culture Collection (CCTCC) on 26 Jun. 2023 under the accession number CCTCC NO: M 20231099. The Lacticaseibacillus rhamnosus FMBL L23004 CNN strain has the effect of inhibiting the activity of α-glucosidase, α-amylase, and dipeptidyl peptidase IV. Administration of the strain can significantly lower oral glucose tolerance and insulin tolerance in Type 2 Diabetes Mellitus (T2DM) mice, reduce abnormal postprandial blood glucose fluctuations, decrease fasting blood glucose levels, lower fasting insulin levels, reduce blood lipid levels, and alleviate insulin resistance. The strain significantly improves the composition of the gut microbiome, increases the abundance and diversity of beneficial gut bacteria, and reduces the relative abundance of pathogenic bacteria.

### CROSS-REFERENCE TO RELATED APPLICATIONS

This application claims priority to Chinese Patent Application No. 202411727413.X, filed on Nov. 28, 2024, which is incorporated herein by reference in its entirety.

### REFERENCE TO SEQUENCE LISTING

This application contains a Sequence Listing filed electronically as an XML file named “Seq.xml”, created on Oct. 21, 2025, with a size of 2,945 bytes. The Sequence Listing is incorporated herein by reference.

### TECHNICAL FIELD

The present disclosure belongs to the technical field of probiotics, and specifically relates to a use of Lacticaseibacillus rhamnosus FMBL L23004 CNN in the preparation of a medicament for preventing or treating diabetes mellitus.

### BACKGROUND

Diabetes mellitus, which is categorized into Type 1 diabetes mellitus and Type 2 diabetes mellitus (T2DM), represents one of the largest global health challenges in the 21st century. Type 1 diabetes mellitus, also known as insulin-dependent diabetes mellitus, predominantly occurs in children and adolescents, with a relatively acute onset. Patients with absolute insulin deficiency are prone to ketoacidosis and require insulin therapy. T2DM constitutes the majority of diabetes mellitus cases. Early symptoms of T2DM may be insidious, and the condition is often detected during physical examinations or upon the emergence of complications. The onset of T2DM is associated with genetic factors on one hand, and with poor lifestyle choices on the other hand. T2DM can be managed through diet, exercise, oral hypoglycemic agents, and insulin therapy. Current therapeutic agents for diabetes mellitus include biguanides, sulfonylureas, non-sulfonylureas, and thiazolidinediones, among others. These agents achieve blood glucose-lowering purposes through mechanisms such as promoting insulin secretion, improving glucose metabolism, ameliorating insulin resistance, and promoting glycogen synthesis. However, these drugs are accompanied by a series of side effects, such as hypoglycemia, gastrointestinal discomfort, obesity, skin pruritus, weight gain, and liver and kidney damage. Consequently, finding “economical, natural, and safe” substances to replace drugs for alleviating or treating diabetes mellitus has become a technical problem urgently needing resolution by those skilled in the art.

Many crucial human functions depend on the gut microbiota. Alterations in the composition, diversity, and metabolites of the gut microbiota can trigger a series of physiological disorders. Studies have found differences in the gut microbiota composition between patients with T2DM and healthy individuals. For instance, the abundance of some beneficial bacteria, such as Bifidobacterium and Lactobacillus, may decrease, while the abundance of some harmful bacteria, such as opportunistic pathogens, may increase. Factors including long-term hyperglycemia, changes in dietary structure, and drug therapy may alter the function of the gut microbiota, leading to gut microbiota dysbiosis, which consequently reduces its fermentation capacity and alters metabolite production. The gut microbiota can ferment dietary fibers to produce short-chain fatty acids (SCFAs), such as acetate, propionate, and butyrate. These SCFAs can stimulate intestinal L-cells to secrete incretins such as Glucagon-Like Peptide-1 (GLP-1) and Peptide YY (PYY), thereby promoting insulin secretion, inhibiting glucagon secretion, and regulating blood glucose levels. Gut microbiota dysbiosis can also increase intestinal permeability, allowing harmful substances like endotoxins to enter the bloodstream, triggering chronic low-grade inflammation and consequently reducing insulin sensitivity. These phenomena indicate that microbial intervention is an important potential pathway for treating or alleviating T2DM.

Inspired by the traditional Chinese medicine theory of “medicinal and edible homology,” the application of probiotics has been endowed with new significance. As an auxiliary therapeutic approach, probiotics are used to create symbiotic probiotic products, such as co-fermentation with traditional Chinese herbs or co-fermentation with fruits and vegetables. The fermentation products serve as functional foods for alleviating T2DM. For example, the Chinese patent invention CN116064285A discloses a Lacticaseibacillus rhamnosus strain ZJUIDSO7 capable of inhibiting α-amylase, α-glucosidase, and Dipeptidyl Peptidase IV (DPP-IV) activity. The Chinese patent invention CN117487692A discloses a Ligilactobacillus gasseri strain FLG-219 possessing hypoglycemic effects.

The research group to which the inventors belong has long been dedicated to research in the field of probiotics. In previous studies, the strain Lacticaseibacillus rhamnosus FMBL L23004 CNN was isolated from human feces and was found to possess antibacterial effects. This strain was subsequently filed as a Chinese patent invention (Application Number 202311683559.4), which has now been granted. Building upon this prior research, the inventors unexpectedly discovered that this strain also exhibits hypoglycemic effects and has the ability to inhibit the activity of α-glucosidase, α-amylase, and Dipeptidyl Peptidase IV. Therefore, the present disclosure is filed to seek protection for the strain Lacticaseibacillus rhamnosus FMBL L23004 CNN.

### SUMMARY

A primary object of the present disclosure is to provide a use of Lacticaseibacillus rhamnosus FMBL L23004 CNN in the preparation of a medicament for preventing or treating diabetes mellitus, wherein the Lacticaseibacillus rhamnosus FMBL L23004 CNN was deposited with the China Center for Type Culture Collection (CCTCC) on 26 Jun. 2023 under the accession number CCTCC NO: M 20231099.

Further, the diabetes mellitus is type 2 diabetes mellitus.

Further, the medicament is for reducing blood glucose levels.

Further, the medicament is for improving insulin resistance.

Further, the Lacticaseibacillus rhamnosus FMBL L23004 CNN exerts the therapeutic effect by inhibiting the activity of α-amylase, α-glucosidase, and/or dipeptidyl peptidase IV.

Further, the medicament is for preventing or treating gut microbiota dysbiosis in a patient with diabetes mellitus.

Further, the medicament is for preventing or treating metabolic dysfunction in a patient with diabetes mellitus.

The present disclosure further provides a pharmaceutical composition, including the Lacticaseibacillus rhamnosus FMBL L23004 CNN, and one or more pharmaceutically acceptable carriers.

Further, a dosage form thereof is one or more selected from the group consisting of a tablet, a capsule, a granule, a powder, a pill, a patch, and a suppository.

The beneficial effects of the present disclosure are: (1) The present disclosure provides a new use of Lacticaseibacillus rhamnosus FMBL L23004 CNN in the preparation of a medicament for treating diabetes mellitus. Results show that both the fermentation supernatant and the intracellular extract possess the effect of inhibiting the activity of α-glucosidase, α-amylase, and dipeptidyl peptidase IV. The inhibition rates against α-glucosidase and α-amylase are 18.62±1.74% and 67.22±5.37%, respectively, and the inhibition rate against DPP-IV is 70.31±1.49%. (2) The Lacticaseibacillus rhamnosus FMBL L23004 CNN can significantly lower oral glucose tolerance and insulin tolerance in T2DM mice, reduce abnormal postprandial blood glucose fluctuations, decrease fasting blood glucose levels, lower fasting insulin levels, and reduce blood lipid levels, thereby alleviating insulin resistance and achieving blood glucose-lowering effects. (3) The Lacticaseibacillus rhamnosus FMBL L23004 CNN also significantly affects the composition of the gut microbiota, increases the abundance and diversity of beneficial bacterial communities in the gut of T2DM mice, and reduces the relative abundance of pathogenic bacteria. (4) The Lacticaseibacillus rhamnosus FMBL L23004 CNN can ameliorate metabolic disorders caused by diabetes mellitus by promoting energy metabolism, carbohydrate metabolism, cofactor metabolism, and vitamin metabolism in T2DM mice.

### BRIEF DESCRIPTION OF THE DRAWINGS

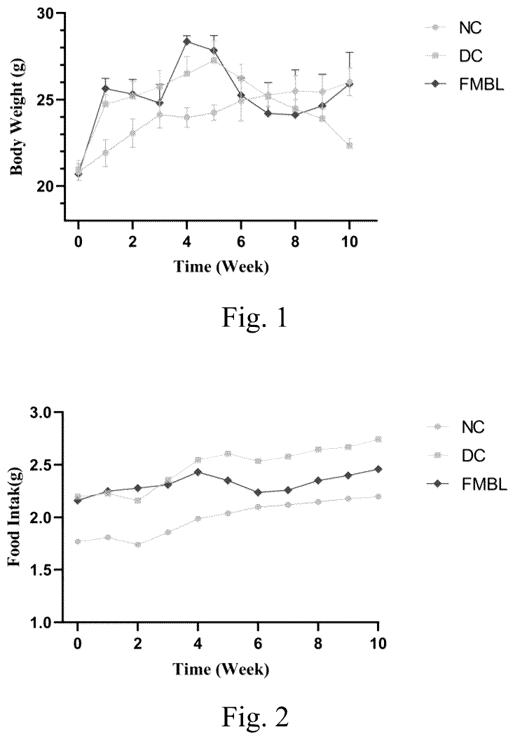

FIG. 1 shows the body weight of mice in each group.
Note: NC represents the normal control group; DC represents the model control group; FMBL represents the probiotic intervention group.

FIG. 2 shows the food intake of mice in each group.
Note: NC represents the normal control group; DC represents the model control group; FMBL represents the probiotic intervention group.

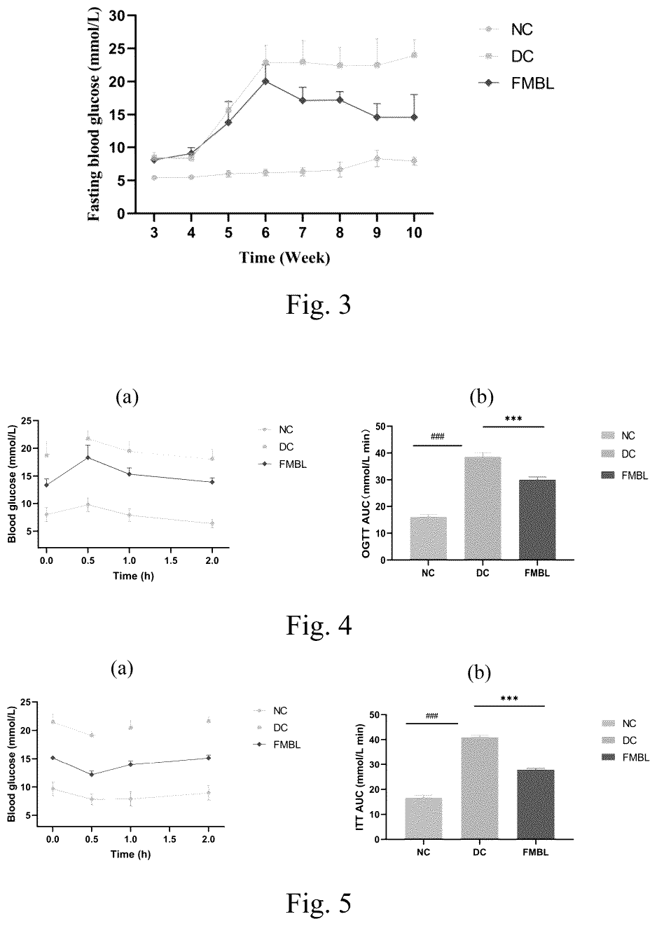

FIG. 3 shows the fasting blood glucose levels of mice in each group.
Note: NC represents the normal control group; DC represents the model control group; FMBL represents the probiotic intervention group.

FIG. 4 shows the Oral Glucose Tolerance Test (OGTT) and the Area Under the Curve (AUC) after 4 weeks of gavage intervention in mice from each group.
Note: NC represents the normal control group; DC represents the model control group; FMBL represents the probiotic intervention group; (a) shows the oral glucose tolerance plot; (b) shows the bar graph of the area under the oral glucose tolerance curve; **P<0.01 and *P<0.05 indicate a statistically significant difference compared with the model control group; ##P<0.01 and #P<0.05 indicate a statistically significant difference compared with the normal control group.

FIG. 5 shows the Insulin Tolerance Test (ITT) and AUC after 4 weeks of gavage intervention in mice from each group.
Note: NC represents the normal control group; DC represents the model control group; FMBL represents the probiotic intervention group; (a) shows the insulin tolerance plot; (b) shows the bar graph of the area under the insulin tolerance curve; **P<0.01 and *P<0.05 indicate a statistically significant difference compared with the model control group; ##P<0.01 and #P<0.05 indicate a statistically significant difference compared with the normal control group.

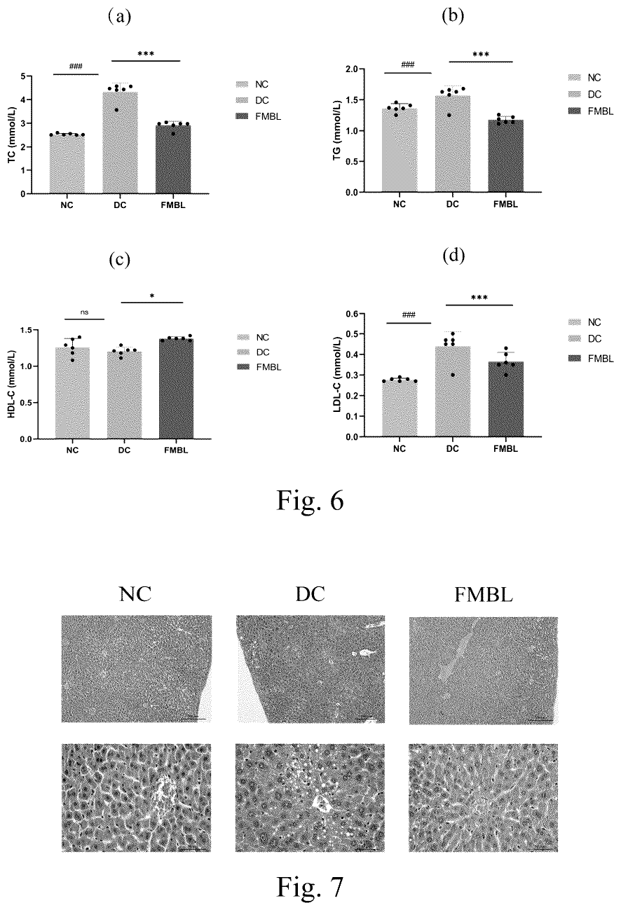

FIG. 6 shows the four blood lipid parameters after 4 weeks of gavage intervention in mice from each group.
Note: NC represents the normal control group; DC represents the model control group; FMBL represents the probiotic intervention group; (a) shows the bar graph of cholesterol (TC) content; (b) shows the bar graph of total triglyceride (TG) content; (c) shows the bar graph of high-density lipoprotein cholesterol (HDL-C) content; (d) shows the bar graph of low-density lipoprotein cholesterol (LDL-C) content; **P<0.01 and *P<0.05 indicate a statistically significant difference compared with the model control group; ##P<0.01 and #P<0.05 indicate a statistically significant difference compared with the normal control group.

FIG. 7 shows the hepatocyte morphology after 4 weeks of gavage intervention in mice from each group.
Note: NC represents the normal control group; DC represents the model control group; FMBL represents the probiotic intervention group.

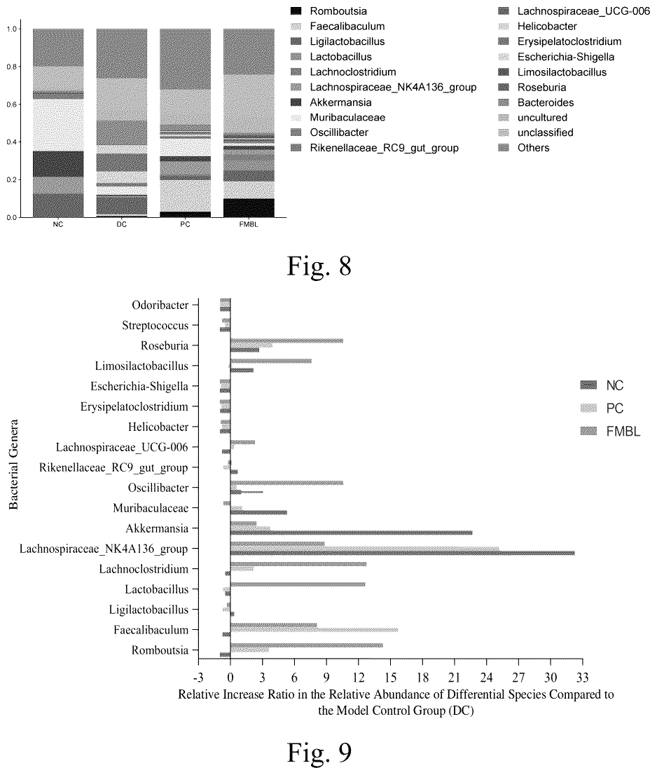

FIG. 8 shows the analysis of the gut microbiota composition at the genus level in mice from each group.
Note: NC represents the normal control group; DC represents the model control group; PC represents the acarbose positive control group; FMBL represents the probiotic intervention group.

FIG. 9 shows the relative change ratio in the relative abundance of differential species for each intervention group compared to the model control group (DC).
Note: NC represents the normal control group; DC represents the model control group; PC represents the acarbose positive control group; FMBL represents the probiotic intervention group.

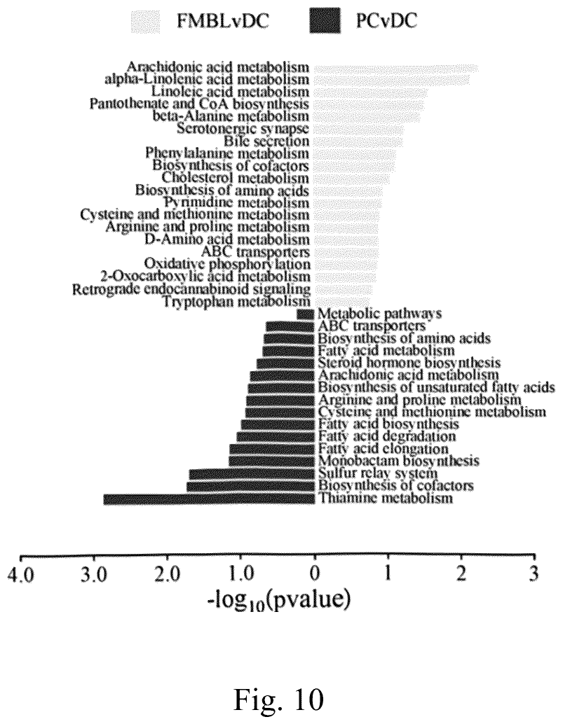

FIG. 10 shows the Kyoto Encyclopedia of Genes and Genomes (KEGG) enrichment pathway analysis of differential fecal metabolites in mice from the probiotic group (FMBL) versus the model control group (DC), and the positive control group (PC) versus the model control group (DC).
Note: DC represents the model control group; PC represents the acarbose positive control group; FMBL represents the probiotic intervention group.

### DETAILED DESCRIPTION OF THE EMBODIMENTS

The following embodiments facilitate a better understanding of the present disclosure but do not limit the scope of the present disclosure. The experimental methods in the following examples, unless otherwise specified, are conventional laboratory methods. The experimental materials used in the following examples, unless otherwise specified, are conventional biochemical reagents.

The formulations of the culture media used are as follows:

MRS liquid medium (1 L): peptone 10 g; beef extract 10 g; yeast extract 5 g; glucose 20 g; Tween 80 1 mL; K₂HPO₄ 2 g; sodium acetate 5 g; diammonium hydrogen citrate 2 g; MgSO₄·7H₂O 0.58 g; MnSO₄·4H₂O 0.25 g; deionized water 1000 mL.

LAMVAB solid medium (1 L): peptone 10 g; beef extract 8 g; yeast extract 4 g; glucose 20 g; Tween 80 1 mL; K₂HPO₄ 2 g; sodium acetate 5 g; diammonium hydrogen citrate 2 g; MgSO₄·7H₂O 0.2 g; MnSO₄·4H₂O 0.05 g; agar 20 g; L-cysteine hydrochloride 0.5 g; vancomycin hydrochloride 20 mg; sterilized at 115° C. for 20 minutes.

### Example 1: Hypoglycemic Effect of Lacticaseibacillus rhamnosus FMBL L23004 CNN

### 1. Bacterial Strains

Test strain: Lacticaseibacillus rhamnosus FMBL L23004 CNN was isolated from a human fecal sample. The strain was deposited with the CCTCC on 26 Jun. 2023 under the accession number CCTCC NO: M 20231099. The deposition address is: Wuhan University, Wuhan, China. Telephone: (027)-68754052, E-mail: cctcc@whu.edu.cn.

Control strain: Lacticaseibacillus rhamnosus GG was purchased from the China General Microbiological Culture Collection Center.

The bacterial strains were streaked on MRS agar plates for activation 2-3 times. A single colony was picked and inoculated into MRS liquid medium for expansion culture at 37° C. for 16 h. The bacterial culture was then inoculated at 2% (v/v) inoculation volume into MRS liquid medium and cultured at 37° C. for 16 h to obtain the bacterial fermentation broth.
(1) Cell-Free Supernatant (CFS): The fermentation broth was centrifuged at 6000 r/min and 4° C. for 10 minutes, then filtered through a 0.22 m aqueous filter membrane. The supernatant was collected and reserved for use.
(2) Cell-Free Extract (CFE): The bacterial cells were washed three times with PBS (Phosphate Buffered Saline). The Optical Density (OD) value was adjusted to 1.0. The cells were disrupted twice using a cell disruptor in expert mode. The solution was centrifuged at 4° C. and 8000 rpm for 20 minutes, then filtered through a 0.22 m filter membrane. The supernatant was collected.

### 2. Determination of α-Amylase Inhibitory Activity

A 125 μL sample solution was mixed with an equal volume of a 1 mg/mL α-amylase solution and incubated in a constant temperature water bath at 37° C. for 15 minutes. Then, the reaction mixture was added to 250 L of a 1% soluble starch solution at 37° C. and reacted at 37° C. for 15 minutes. Subsequently, 500 μL of 3,5-Dinitrosalicylic acid (DNS) solution was added. The mixture was heated in a boiling water bath for 5 minutes, then rapidly cooled to room temperature. After diluting 20-fold and allowing the diluted mixture to stand until reaching room temperature, the absorbance was measured at 540 nm. A PBS solution (0.1 mol/L, pH=6.8) was used as a blank control for both the α-amylase solution and the test sample.

$$α - Amylase inhibition rate = [ 1 - ( A - B ) / ( C - D ) ] * 100 \%$$

Where: A represents the sample group, containing the sample solution and the α-amylase solution. B represents the sample control group, containing the sample solution but no α-amylase solution. C represents the control group, containing no sample solution but containing the α-amylase solution. D represents the blank control group, containing no sample solution and no α-amylase solution.

### 3. Determination of α-Glucosidase Inhibitory Activity

A mixture of 50 μL of the fermentation supernatant and 100 μL of a 1 U/mL α-glucosidase solution (prepared in 0.1 mol/L phosphate buffer, pH 6.8) was added to a test tube, mixed well, and reacted at a constant temperature of 37° C. for 10 minutes. Then, 50 μL of a 5 mmol/L pNPG solution (prepared in 0.1 mol/L phosphate buffer, pH 6.8) was added, mixed well, and the reaction continued at a constant temperature of 37° C. for 30 minutes. Subsequently, 1 mL of a 0.1 mol/L Na2CO3 solution was added, and the absorbance was measured at 400 nm. PBS was used instead of the sample and α-glucosidase as the control.

$$α - Glucosidase inhibition rate = [ 1 - ( A - B ) / ( C - D ) ] * 100 \%$$

Where: A represents the sample group, containing the sample solution and the α-glucosidase solution. B represents the sample control group, containing the sample solution but no α-glucosidase solution. C represents the control group, containing no sample solution but containing the α-glucosidase solution. D represents the blank control group, containing no sample solution and no α-glucosidase solution.

TABLE 1
In Vitro Inhibitory Activity of Lacticaseibacillus rhamnosus
Strains Against α-Amylase and α-Glucosidase

| | α-Amylase Inhibitory | | α-Glucosidase Inhibitory | |
|---|---|---|---|---|
| | Activity (%) | | Activity (%) | |
| Strain | CFS | CFE | CFS | CFE |
| Lacticaseibacillus | 67.22 ± | 9.01 ± | 18.62 ± | 3.51 ± |
| rhamnosus FMBL | 5.37^{a} | 0.4^{a} | 1.74^{a} | 0.27^{a} |
| L23004 CNN | | | | |
| Lacticaseibacillus | 43.33 ± | −21.49 ± | 4.23 ± | −19.21 ± |
| rhamnosus GG | 2.03^{b} | 1.02^{b} | 1.6b^{b} | 1.74^{b} |

Note:
Different superscript letters (a, b, c) represent statistically significant differences at P < 0.05.

The results presented in Table 1 indicate that the CFE of the strain Lacticaseibacillus rhamnosus GG not only failed to inhibit but actually promoted the activity of both enzymes. In contrast, both the CFS and the CFE of Lacticaseibacillus rhamnosus FMBL L23004 CNN demonstrated a considerable ability to inhibit the activity of α-amylase and α-glucosidase.

### 4. Determination of DPP-IV Inhibitory Activity

Into a 96-well microplate, 25 μL of Glycine-p-nitroanilide (0.2 mM) and 25 μL of the CFS were added and pre-incubated at 37° C. for 10 minutes. Subsequently, 50 μL of DPP-IV (0.01 U/mL) was added and incubated at 37° C. for 60 minutes. The reaction was then terminated by adding 100 μL of sodium acetate buffer (1 M, pH 4.0). The absorbance of the sample was measured at 405 nm. In the reaction system, a PBS solution (0.1 mol/L, pH 6.8) was used as a blank control for both the DPP-IV solution and the test sample.

$$DPP - IV inhibition rate = [ 1 - ( A - B ) / ( C - D ) ] * 100 \%$$

Where: A represents the measured absorbance value for the well containing both the DPP-IV solution and the test sample. B represents the measured absorbance value for the well containing the test sample but no DPP-IV solution. C represents the measured absorbance value for the well containing the DPP-IV solution but no test sample. D represents the measured absorbance value for the well containing neither the DPP-IV solution nor the test sample.

TABLE 2
DPP-IV Inhibition Rate of Bacterial Fermentation Supernatants

| Group | DPP-IV Inhibition Rate (%) |
|---|---|
| Lacticaseibacillus rhamnosus FMBL | 70.31 ± 1.49^{a} |
| L23004 CNN | |
| Lacticaseibacillus rhamnosus GG | 8.33 ± 0.84^{b} |

Note:
Different superscript letters (a, b, c) represent statistically significant differences at P < 0.05.

The results presented in Table 2 indicate that Lacticaseibacillus rhamnosus FMBL L23004 CNN demonstrates significant application potential in lowering blood glucose and improving diabetes mellitus conditions. The strain can be used in the preparation of products for lowering blood glucose, improving obesity, and managing diabetes mellitus.

### Example 2: Ameliorative Effect of Lacticaseibacillus rhamnosus FMBL L23004 CNN on T2DM Mice

### 1. Preparation of Bacterial Suspension

The bacterial strain was streaked on MRS agar plates for activation 2-3 times. A single colony was picked and inoculated into MRS liquid medium for expansion culture at 37° C. for 16 h. The bacterial culture was then inoculated at 2% (v/v) inoculation volume into MRS liquid medium and cultured at 37° C. for 18 h to obtain the bacterial fermentation broth. The Lacticaseibacillus rhamnosus FMBL L23004 CNN fermentation broth was centrifuged (4000 r/min, 3 min, 4° C.), and the supernatant was discarded. The bacterial pellet was washed twice with 0.9% sterile physiological saline. The bacterial suspension concentration was adjusted to 1×10⁹ CFU/mL for subsequent use.

### 2. Animal Experiment

### 2.1 Establishment of T2DM Mouse Model and Grouping

A total of twenty-four male C57BL/6J mice aged 8 weeks (20 g±1 g) were selected and purchased from Henan Skobes Biotechnology Co., Ltd. Standard maintenance feed and high-fat diet H10060 were purchased from Xinjiang Hengchao Biotechnology Co., Ltd. After one week of dietary adaptation, the mice were randomly divided into a normal control group (NC, 6 mice) and an experimental group (18 mice). The normal control group was fed a standard diet, while the experimental group was fed the high-fat diet. After four weeks on the respective diets, the mice in the experimental group received multiple low-dose intraperitoneal injections of 45 mg/kg Streptozotocin (STZ) (prepared in citric acid-sodium citrate buffer, pH 4.5; intraperitoneal injection volume 0.1 mL/10 g body weight). One week after the STZ injection, mice with Fasting Blood Glucose (FBG) levels exceeding 16.5 mmol/L were considered successful models.

After successful model establishment, the experimental group was divided into the model control group (DC), the positive control group (PC), and the probiotic intervention group (FMBL), with 6 mice in each group.

The normal control group and the model control group received gavage with an equal volume of 0.9% sterile physiological saline for 4 weeks. The positive control group received acarbose at a dosage of 200 mg/kg/day. The probiotic intervention group received Lacticaseibacillus rhamnosus FMBL L23004 CNN at a dosage of 10 mg/kg/day based on mouse body weight.

The body weight of the mice in each group was measured weekly using an electronic balance, and food intake was recorded. During the intervention period, the FBG of the mice was measured at a fixed time each week.

### 2.2 Euthanasia of T2DM Mice and Tissue Collection

One week before the end of the experiment, feces from each mouse were collected into 2 mL sterile centrifuge tubes and stored at −80° C. for subsequent 16S rRNA and non-targeted metabolomics analysis. Before euthanasia, all mice were fasted but allowed free access to sterile distilled water. After euthanasia, blood was collected from the orbital sinus of all mice. The blood was allowed to stand at room temperature for 1 hour to coagulate, then centrifuged at 4° C. and 3500 rpm for 45 minutes. The mouse serum was collected and stored at −80° C. for subsequent indicator analysis. The livers of the dissected mice were collected; a portion of the liver was placed in 4% paraformaldehyde solution for tissue fixation, and the remaining portion was flash-frozen in liquid nitrogen and stored at −80° C. for subsequent use.

### 2.3 Oral Glucose Tolerance Test

One week before the end of the experiment, an Oral Glucose Tolerance Test (OGTT) was performed on the mice. The mice were fasted for 12 hours with free access to water. Body weight was measured before the test to determine the glucose gavage dose. The mice were then administered a glucose solution at 2 g/kg body weight by oral gavage. Blood glucose levels were measured at 30, 60, 90, and 120 minutes after administration. Blood glucose curves were plotted, and the AUC for blood glucose was calculated using GraphPad Prism software (version 8.0.2).

### 2.4 Insulin Tolerance Test

One week before the end of the experiment, an Insulin Tolerance Test (ITT) was performed on the mice. The mice were fasted for 12 hours with free access to water. Body weight was measured before the test to determine the insulin injection dose. The mice were then intraperitoneally injected with insulin at 1 U/kg body weight. Blood glucose levels were measured at 30, 60, 90, and 120 minutes after injection. Blood glucose curves were plotted, and the AUC for blood glucose was calculated using GraphPad Prism software (version 8.0.2).

### 2.5 Determination of Insulin-Related Indices

Mouse serum was collected. Fasting Insulin (FINS) levels in the serum of fasted mice were determined using an Enzyme-Linked Immunosorbent Assay (ELISA) kit. The Insulin Sensitivity Index (ISI) was calculated as the natural logarithm of the reciprocal of the product of FBG (measured before the end of the experiment) and FINS: ISI=ln(1/(FBG×FINS)). The Homeostatic Model Assessment of Insulin Resistance (HOMA-IR) was calculated as: HOMA-IR=(FBG×FINS)/22.5. The Homeostatic Model Assessment of β-cell function (HOMA-β) was calculated as:

$$H OMA - β = ( FINS \times 20 ) / ( FBG - 3.5 ) .$$

### 2.6 Serum Parameter Measurements

Serum lipid metabolism levels were detected using a Hitachi automated biochemical analyzer. The measured parameters included Total Cholesterol (TC), Triglycerides (TG), High-Density Lipoprotein Cholesterol (HDL-C), and Low-Density Lipoprotein Cholesterol (LDL-C).

### 2.7 Histopathological Sectioning

Liver tissue samples collected from the dissected mice were fixed in 4% paraformaldehyde at 4° C. for 48 hours. The tissues were then dehydrated through a graded ethanol series, cleared in xylene, embedded in paraffin, sectioned, and dried. The liver sections were stained with Hematoxylin and Eosin (H&E), observed, and photographed under a microscope at 400× magnification.

### 3. Data Processing

All data are expressed as mean+standard deviation (SD). Significant differences between groups were determined using one-way Analysis of Variance (ANOVA). Statistical significance was set at P<0.05. Statistical analysis was performed using GraphPad Prism software (version 8.0.2).

### 4. Results

Sustained body weight loss is one of the typical symptoms of T2DM. As shown in FIG. 1, the normal control group was fed a standard diet, while the T2DM model mice were fed a high-fat diet. The body weight of the normal control group mice increased slowly over the feeding period, eventually reaching 25.1 g; the body weight of the mice fed the high-fat diet increased sharply during the modeling process, reaching 25.0 g by the fifth week. After three consecutive STZ injections in the fifth week, the body weight of the mice in the intervention groups began to decrease, except for the normal control group; this body weight change is consistent with the clinical weight characteristics of patients with diabetes mellitus, indicating that the diabetes mellitus modeling was successful from the perspective of body weight changes. After successful model induction, the body weight of the model control group mice continued to decrease, whereas the body weight of the mice in the probiotic intervention group showed a trend of decreasing first and then increasing. By the 10th week, the body weight of the mice in the probiotic intervention group was essentially the same as that of the normal control group, reaching 25.0 g. This indicates that intervention with Lacticaseibacillus rhamnosus FMBL L23004 CNN can effectively ameliorate the symptom of body weight loss in T2DM mice.

As shown in FIG. 2, throughout the experimental period, the food intake of the mice in the model control group and the probiotic intervention group was significantly higher than the food intake of the mice in the normal control group. After the STZ injection in the fifth week, compared with the model control group, the food intake of the mice in the probiotic intervention group decreased significantly and gradually approached the food intake level of the normal control group. This indicates that administration of Lacticaseibacillus rhamnosus FMBL L23004 CNN alleviated the symptoms of T2DM in the mice and reduced their food intake.

Elevated FBG levels are the most representative characteristic of T2DM, and the level of FBG is also the gold standard for judging the success of the establishment of the diabetic mouse model. After the STZ injection in the fifth week, the model was considered successful when the mice had FBG values greater than 16.5 mmol/L for three consecutive days. The results are shown in FIG. 3. Throughout the experimental period, the blood glucose of the normal control group mice dynamically fluctuated within the range of 5-9 mmol/L. The FBG levels of the model control group ranged between 20 and 25 mmol/L, indicating successful model induction. Compared with the model control group, the blood glucose levels of the T2DM mice decreased significantly as the intervention period progressed after the administration of the probiotic. This result indicates that intervention with Lacticaseibacillus rhamnosus FMBL L23004 CNN can effectively improve/lower the blood glucose levels in T2DM mice.

The OGTT results are shown in FIG. 4. After successful model induction, the AUC for the model control group increased significantly. Compared with the model control group, the AUC for the probiotic intervention group decreased significantly. This indicates that the model control group mice require a longer recovery time for their ability to regulate blood glucose, whereas intervention with Lacticaseibacillus rhamnosus FMBL L23004 CNN can significantly lower the oral glucose tolerance in T2DM mice and effectively improve/restore the T2DM mice's ability to regulate blood glucose.

The results are shown in FIG. 5. After successful model induction, the AUC for the model control group increased significantly. Compared with the model control group, the probiotic intervention significantly reduced the insulin tolerance in T2DM mice. This indicates that intervention with Lacticaseibacillus rhamnosus FMBL L23004 CNN can effectively reduce/alleviate insulin resistance in T2DM mice.

As shown in Table 3, the FINS and HOMA-IR indices were significantly elevated in the model control group mice, indicating that the model control group mice developed insulin resistance, could not maintain normal blood glucose levels, and had impaired blood glucose regulation. Compared with the model control group, the FBG, FINS, and HOMA-IR indices were significantly decreased in the probiotic intervention group mice; the ISI and HOMA-β indices were significantly increased.

Intervention with Lacticaseibacillus rhamnosus FMBL L23004 CNN can significantly increase the insulin sensitivity in T2DM mice, improve the insulin resistance in T2DM mice, promote the proliferation of pancreatic β-cells, and consequently restore their blood glucose regulation ability.

TABLE 3
Insulin-Related Indices

| | FINS | FBG | | | |
|---|---|---|---|---|---|
| Group | (mIU/L) | (mmol/L) | ISI | HOMA-IR | HOMA-β |
| Normal | 131.6 ± | 7.56 ± | −6.9 ± | 44.17 ± | 667 ± |
| Control | 5.11^{cd} | 0.78^{cd} | 0.1^{a} | 4.27^{cd} | 125.7^{a} |
| Group (NC) | | | | | |
| Model | 231.92 ± | 23.44 ± | −8.6 ± | 241.76 ± | 234.53 ± |
| Control | 3^{a} | 2.04^{a} | 0.1^{c} | 23.17^{a} | 23.47^{d} |
| Group (DC) | | | | | |
| Acarbose | 134.82 ± | 8.62 ± | −7.05 ± | 51.65 ± | 540.96 ± |
| Positive | 3.54^{c} | 0.88^{c} | 0.11^{ab} | 5.62^{c} | 97.9^{b} |
| Control | | | | | |
| Group (PC) | | | | | |
| Probiotic | 174.25 ± | 14.98 ± | −7.87 ± | 116.09 ± | 305.31 ± |
| Intervention | 4.17^{b} | 1.04^{b} | 0.08^{b} | 9.84^{b} | 24.92^{c} |
| Group | | | | | |
| (FMBL) | | | | | |

As shown in FIG. 6, the levels of TC, TG, and LDL-C in the model control group mice were significantly higher than those in the normal control group. This indicates that the abnormal glucose metabolism in the diabetic mice indeed caused abnormal cholesterol metabolism. The levels of TC, TG, and LDL-C in the probiotic intervention group mice were close to those in the normal control group and significantly lower than those in the model control group, while the HDL-C level was significantly higher than that in the model control group. The above results indicate that intervention with Lacticaseibacillus rhamnosus FMBL L23004 CNN can effectively regulate the blood lipid metabolism in T2DM mice, improve the levels of TG, TC, HDL-C, and LDL-C in diabetic mice, and have the potential to reduce the risk of cardiovascular diseases and thrombosis.

The histopathological results of the mouse livers are shown in FIG. 7. Excessive accumulation and deposition of lipids in the liver caused liver cells in the histological sections of the model control group mice to contain lipid droplet vacuoles of varying sizes, and the steatosis was more severe, indicating that the mice exhibited symptoms of impaired liver fat metabolism to a certain extent. Compared with the model control group, the number and volume of lipid droplet vacuoles decreased in the liver sections of the probiotic intervention group mice, and the liver cell structure became relatively clear and intact. This indicates that Lacticaseibacillus rhamnosus FMBL L23004 CNN can significantly reduce hepatic lipid accumulation and steatosis in T2DM mice and can exhibit a favorable restorative and protective effect on the liver.

### Example 3: Effect of Lacticaseibacillus rhamnosus FMBL L23004 CNN on the Gut Microbiota of T2DM Mice

### 1. Analysis of Gut Microbial Diversity and Community Structure Composition

Before the end of the experiment, fecal samples were obtained by stimulating the mice's anal regions. Feces from mice in each group were collected using sterile forceps, placed into 2 mL sterilized cryotubes, and stored in a −80° C. ultra-low temperature freezer for analysis of the mice's gut microbiota structure.

Total bacterial DNA was extracted from the feces according to the instructions of the E.Z.N.A. Stool DNA Kit. The V3-V4 hypervariable region of the 16S rRNA gene was selected for PCR amplification using specific primers with Barcodes:

338F(SEQ ID NO: 1):
5′-barcode-ACTCCTACGGGAGGCAGCAG-3′;
806R(SEQ ID NO: 2):
5′-GGACTACHVGGGTWTCTAAT-3′

PCR products were pooled in equal amounts based on concentration. After thorough mixing, the PCR products were purified by electrophoresis on a 2% agarose gel in 1× TAE buffer. Bands in the size range of 400-450 bp (target bands) were excised, recovered, and further purified using the AxyPrep DNA Gel Extraction Kit. The DNA concentration in the extracted PCR products was quantified using the QuantiFluor™ dsDNA Assay Kit. Libraries were constructed using the Ultra™ DNA Library Prep Kit for Illumina. After qualification by Qubit quantification and library testing, the constructed libraries were sequenced on the MiSeq platform. The raw sequencing data were first spliced and filtered to obtain effective data. Effective data were subjected to OTU (Operational Taxonomic Unit) clustering and species classification analysis. Based on the OTU clustering results, representative sequences of each OTU were annotated for species. According to the species annotation results, stacked bar charts of species relative abundance were generated. LEfSe (Linear Discriminant Analysis Effect Size) was used to test for significant differences in species composition among grouped samples.

### 2. Results

2.1 Effect of Lacticaseibacillus rhamnosus FMBL L23004 CNN on the Gut Microbiota Structure Composition in T2DM Mice

As shown in FIG. 8, compared with the model control group, the relative abundances of various beneficial bacteria, such as Romboutsia (9.9%), Faecalibaculum (9.14%), Ligilactobacillus (5.72%), Lactobacillus (5.33%), Akkermansia (1.99%), and Lachnoclostridium (3.13%), were significantly increased in the gut of mice from the probiotic intervention group. As shown in FIG. 9, compared with the model control group, the relative abundances of genera including Romboutsia, Akkermansia, Faecalibaculum, Lactobacillus, and Roseburia in the gut of mice from the positive control group increased by 3.63-fold, 3.75-fold, 15.72-fold, 0.7-fold, and 3.9-fold, respectively, while the relative abundance of Limosilactobacillus decreased by 0.2-fold. In contrast, the relative abundances of genera including Romboutsia, Akkermansia, Faecalibaculum, Lactobacillus, Limosilactobacillus, and Roseburia in the gut of mice from the probiotic intervention group were significantly increased by 14.3-fold, 2.45-fold, 8.1-fold, 12-fold, 2.2-fold, and 10.6-fold, respectively, compared to the model control group. Compared with the normal control group, the relative abundances of Erysipelatoclostridium (9.41%), Helicobacter (6.21%), Escherichia-Shigella (4.58%), Fusobacterium (0.91%), Streptococcus (1.18%), and Odoribacter (3.64%) were significantly increased in the gut of mice from the model control group. Compared with the model control group, the relative abundances of Erysipelatoclostridium (0.018%), Helicobacter (0.525%), Escherichia-Shigella (0.01%), Fusobacterium (0%), Streptococcus (0.24%), and Odoribacter (0.07%) were significantly decreased in the gut of mice from the probiotic intervention group.

The above results indicate that intervention with Lacticaseibacillus rhamnosus FMBL L23004 CNN significantly affected the composition of the gut microbiota in diabetic mice, significantly increased the number of species and relative abundance of beneficial bacteria in the gut of T2DM mice, and reduced the relative abundance of pathogenic bacteria.

### Example 4: Lacticaseibacillus Rhamnosus FMBL L23004 CNN Ameliorates Metabolic Disorders Induced by T2DM

### 1. Sample Collection and Preparation

One week before the end of the experiment, feces from mice in each group were collected. A threefold volume of methanol solution was added to the feces. The mixture was vortexed for 5 minutes and then centrifuged at 5000 r/min for 10 minutes. The supernatant was collected, passed through a 0.22 m organic filter membrane, and stored for subsequent UPLC-Q-TOFMS analysis.

### 2. Experimental Conditions

### 2.1 Chromatographic Conditions

Chromatographic column: Waters Acquity UPLC BEH C18 column (2.1 mm×50 mm×1.7 m); Column temperature: 40° C.; Injection volume: 5 μL; Mobile phase: A was acetonitrile, B was a 0.1% formic acid aqueous solution; Flow rate: 0.3 mL/min; Gradient elution conditions: 0-1 min (5%-30% A), 1-3 min (30%-50% A), 3-3.6 min (50%-53% A), 3.6-4.2 min (53%-60% A), 4.2-6 min (60%-80% A), 6-8 min (80%-100% A), 8-9 min (100% A).

### 2.2 Mass Spectrometric Conditions

Ion source: Electrospray Ionization (ESI); Ion source temperature: 120° C.; Mass scan range: m/z 100-1000; Both the cone gas and desolvation gas were nitrogen, with flow rates of 50 L/h and 700 L/h, respectively; The desolvation gas temperature was 350° C. In positive ion mode, the capillary voltage was 3.0 kV; in negative ion mode, the capillary voltage was 2.0 kV. In both positive and negative ion modes, the cone voltage and the extractor cone voltage were 40 V and 5.0 V, respectively. A sodium formate solution was used to establish the mass calibration curve; Leucine Enkephalin (LE) was used for real-time mass correction. Argon was used as the collision gas for MS/MS analysis, with low collision energy set at 5 eV and high collision energy ramped from 10 to 25 eV.

### 2.3 Data Analysis

The acquired raw data files were imported into the software Compound Discoverer™ 3.3 (version 3.3.2.31, Thermo, Waltham, USA) for peak detection, filtering, and alignment. Peaks not detected in more than 50% of the Quality Control (QC) samples were filtered out. Missing values for undetected peaks were filled based on the software's Fill Gaps algorithm. Data correction was performed using the total peak area normalization method, resulting in a quantified metabolite list. Differential metabolites were screened based on the Variable Importance in Projection (VIP) value and the P-value. A metabolite was considered to have a statistically significant difference when the VIP was >1 and the P-value was <0.05. Functional pathway enrichment analysis of the screened differential metabolites was performed using MetaboAnalyst 6.0, and visualization was conducted using the KEGG Mapper tool.

### 3. Results

Non-targeted metabolomics analysis was performed on feces from the normal control group, model control group, positive control group, and probiotic intervention group, detecting 1934, 1494, 1551, and 1500 substances, respectively. These substances primarily included organic acids and derivatives, lipids and lipid-like molecules, organoheterocyclic compounds, benzenoids, phenylpropanoids and polyketides, organic oxygen compounds, organic nitrogen compounds, nucleosides and their analogues, alkaloids and derivatives, among others. Analysis of differential metabolites revealed that 898 differential metabolites were identified in the normal control group versus the model control group comparison, among which 304 metabolites were significantly increased and 594 metabolites were significantly decreased. In the positive control group versus model control group comparison, 131 differential metabolites were identified, among which 97 metabolites were significantly increased and 34 metabolites were significantly decreased. In the probiotic intervention group versus model control group comparison, 298 differential metabolites were identified, among which 244 metabolites were significantly increased and 54 metabolites were significantly decreased. These results indicate that the probiotic intervention significantly affected the fecal metabolites in T2DM mice.

Compared with the normal control group, the levels of Apabetalone, Histidine, Cinncassiol A, and Valine in the model control group decreased by 7.26-fold, 9.78-fold, 33.98-fold, and 2.41-fold, respectively, while the levels of Monomethyl Phthalate, 7-ketocholesterol, and 5-Aminovaleric Acid Betaine increased by 33.33-fold, 1.65-fold, and 3.57-fold, respectively. Compared with the model control group, the levels of beneficial substances increased in the positive control group, while the levels of detrimental substances such as Monomethyl Phthalate decreased. Compared with the model control group, the levels of beneficial substances in the probiotic intervention group, including Apabetalone, Histidine, Cinncassiol A, Valylproline, Vitamin B5, N-Acetyltaurine, 3-Hydroxyphenylacetic Acid, Traumatic Acid, and Equisetin, which are beneficial for improving diabetes mellitus, increased by 5.48-fold, 0.63-fold, 7-fold, 0.65-fold, 3.21-fold, 1.05-fold, 3.58-fold, 2.84-fold, 0.9-fold, and 2.6-fold, respectively. Conversely, the levels of Monomethyl Phthalate, 7-ketocholesterol, and 5-Aminovaleric Acid Betaine decreased by 63%, 20%, and 27%, respectively. These findings demonstrate that the probiotic intervention, similar to the positive control, can ameliorate metabolic disorders caused by diabetes mellitus by significantly increasing the levels of beneficial substances and decreasing the levels of detrimental substances in the gut of T2DM mice.

KEGG enrichment analysis was performed on the differential metabolites; the results are shown in FIG. 10. Compared with the model control group, the fecal metabolites of mice in the probiotic intervention group were mainly enriched in metabolic pathways such as amino acid metabolism, lipid metabolism, metabolism of cofactors and vitamins, carbohydrate metabolism, energy metabolism, and nucleotide metabolism, primarily related to the digestive system, endocrine system, and nervous system. Compared with the model control group, the fecal metabolites of mice in the positive control group were mainly enriched in amino acid metabolism, lipid metabolism, and metabolism of cofactors and vitamins. Compared with the positive control group, the probiotic intervention further promoted energy metabolism, carbohydrate metabolism, and metabolism of cofactors and vitamins in T2DM mice by participating in the regulation of the host's nervous system, digestive system, and endocrine system, thereby ameliorating the metabolic disorders induced by diabetes mellitus.

What is claimed is:

1. A use of Lacticaseibacillus rhamnosus FMBL L23004 CNN in the preparation of a medicament for preventing or treating diabetes mellitus, wherein the Lacticaseibacillus rhamnosus FMBL L23004 CNN was deposited with the China Center for Type Culture Collection (CCTCC) on 26 Jun. 2023 under the accession number CCTCC NO: M 20231099.

2. The use according to claim 1, wherein the diabetes mellitus is type 2 diabetes mellitus.

3. The use according to claim 1, wherein the medicament is for reducing blood glucose levels.

4. The use according to claim 1, wherein the medicament is for improving insulin resistance.

5. The use according to claim 1, wherein the Lacticaseibacillus rhamnosus FMBL L23004 CNN exerts the therapeutic effect by inhibiting the activity of α-amylase, α-glucosidase, and/or dipeptidyl peptidase IV.

6. The use according to claim 1, wherein the medicament is for preventing or treating gut microbiota dysbiosis in a patient with diabetes mellitus.

7. The use according to claim 1, wherein the medicament is for preventing or treating metabolic dysfunction in a patient with diabetes mellitus.


SEQUENCE LISTING (2 sequences)

SEQ ID NO: 1 - DNA, 20 bp, synthetic construct
  source 1..20: mol_type = other DNA
actcctacgggaggcagcag

SEQ ID NO: 2 - DNA, 20 bp, synthetic construct
  source 1..20: mol_type = other DNA
ggactachvgggtwtctaat